(19) United States
(12) Patent Application Publication
(10) Pub. No.: US 2017/0000320 A1
(43) Pub. Date: Jan. 5, 2017

(54) ASEPTIC JOINT ASSEMBLY FOR A SURGICAL VISUALIZATION SYSTEM

(71) Applicant: Vantage Surgical Systems, Inc., Irvine, CA (US)

(72) Inventors: Jason Wilson, Los Angeles, CA (US); Andre Bessette, San Mateo, CA (US)

(21) Appl. No.: 14/789,446

(22) Filed: Jul. 1, 2015

(51) Int. Cl. A61B 1/00; A61B 19/08; A61B 19/12; A61B 1/313

(52) U.S. Cl. CPC A61B 1/00149; A61B 1/3132; A61B 19/081; A61B 1/00052; A61B 1/00142; A61B 1/0014; A61B 19/12; A61B 2019/085

(57) ABSTRACT

Embodiments of the present disclosure include an aseptic joint assembly for use in minimally invasive surgical imaging systems implementing a positioning arm. According to some aspects, the aseptic joint assembly includes at least one adaptor having a locking mechanism used to removably attach the adaptor onto an end of the positioning arm of the surgical visualization positioning system and directly or indirectly support at least one or both a display and an imaging device of the visualization system. In some embodiments, controlled rotation about one or more axis can be provided for moving the imaging device via an end effector and in relation to a surgical site. According to additional aspects, a surgical drape can be bonded to at least a portion of an adaptor and is configured to cover at least a portion a length of the positioning arm of the surgical visualization system.

### FIELD OF THE INVENTION

The present invention relates generally to the field of minimally invasive surgery (MIS) and more particularly to improved aseptic medical devices/components for visualization systems used in the surgical procedures.

### BACKGROUND OF THE INVENTION

Unlike in traditional open surgery procedures, with the use of visualization systems, minimally invasive surgeries (MIS) allow practitioners to perform surgical procedures via small incisions for the same common purpose. These MIS controlled procedures are beneficial to the patient in that they result in less damage to biological tissue and thus less pain, scarring and faster recoveries. More importantly, by reducing the incision size and damage to biological tissue, MIS helps minimize the risk of infections, and in most cases, the overall cost of the surgical treatment.

Passive and active visualization systems are currently being used by practitioners to perform MIS. Both of these types of systems can include fiber optic cables, miniature video cameras and instruments which have been redesigned to be handled via tubes inserted in the small incision(s) to enable the practitioner to perform the procedure while indirectly viewing images/video of the surgical site that are transmitted to external monitors. Because MISs are so different than the traditional open surgery procedures, significant learning and practice is required from practitioners, including for example simulated learning cadaveric training, to be able to operate these systems that enable MIS but require indirect visualization of the surgical site.

MIS visualization systems use hand held endoscopes for capturing the surgical site images displayed on the monitor(s). In using hand held endoscopes however, the practitioner often has to give up visualization control to an assistant (assistant surgeon, attending nurse, etc.) to steer the endoscope via verbal instructions from him/her. With instructions from the practitioner, the assistant rotates the endoscope about the surgical incision to view different locations of the surgical area and/or physically translates the endoscope through the incision closer to the tissue to view magnified views.

In order to free the assistant from the camera steering task or retain control by the practitioner, two types of endoscope positioning arms for the MIS visualization systems have been developed. These arms fall into two categories—passive and active. Passive means that there are no actuators. The user must manually move the endoscope to the correct location and then the arm is locked into place maintaining the endoscope viewing directions. Active means that actuators are attached to the arm articulating joints, allowing it to be teleoperated via some human machine interface (HMI) or in a robotic fashion. For both active and passive types there are two common vital properties in need of much improvement to facilitate learning and operating the use of these systems in the operating room—(1) improved intuitive user interface, and (2) minimizing intrusiveness to the surgeon in the surgical field (i.e. low profile).

With both active and passive systems, when operating an endoscope positioning arm during surgery the surgeon, or assistant, may need to intermittently move the endoscope to a particular location to image the appropriate surgical field tissues. Once in location, the surgeon or assistant may return to other critical tasks while the positioning arm holds the endoscope in place. In the case of the surgeon steering the endoscope, any time spent interacting with the positioning arm is time taken away from surgical task(s) and requires him/her to re-orient himself/herself with the new perspective resulting from the movement and being projected by the monitor, which is outside the line of sight of the surgeon. Currently active arms have implemented different control modalities including joystick, voice, and head mounted trackers, for example. Most commercialized versions of these devices have been discontinued or simply not found in many operating rooms due to their difficulty to use. One exception is the da Vinci surgical systems® by Intuitive Surgical™. This device uses the joystick paradigm for remote control. Practitioners have learned and continue to implement this device, at least in part due to the joystick which can control both the surgical instruments and visualization, thus allowing the use of a single HMI for all surgical tasks. However, this system is extremely complex (e.g. due to its cost and constant calibration requirements) and in some MIS procedures where it is preferred for the practitioner to manipulate the instruments directly—the system's practicality is contradicted and often results in an obstruction to the practitioner.

Similarly, the available passive positioning arms also suffer from a cumbersome HMIs. In these system, by nature the operator typically must loosen joints separately from the adjustment of the endoscope resulting in a time consuming process in need of much improvement. Further, some additionally suffer from redundant degrees of freedom which while allowing for versatile positioning of the arm and the endoscope, require additional time, from the surgeon or assistant, to adjust the pose of the arm as well as the position of the endoscope at times resulting in a hazard during surgery.

The center for disease control and prevention (CDC) estimates that in about a third of the approximately 27 million surgical procedures performed in the United States each year a hospital acquired infection occurs resulting in recovery complications, longer hospital stays, and increased costs. Surgical drapes are regularly used to prevent infections by creating barriers between aseptic surgical areas and non-sterile parts or environments. Due to the complexity of many active and passive positioning arms however, proper sterilization may not possible and the use of drapes has been overly cumbersome. For example, using drapes to create a barrier between the non-sterile arm and the surgical field in joints and moving parts can be pose significant challenges. While this could help keep a sterile environment, all articulating joints of the positioning arm must be draped resulting in a cumbersome time consuming process and the drapes can often result in obstructions around the surgical sterile areas. Moreover, the drapes make it difficult to remove the visualization device in case of an emergency and can easily become tangled due to relative rotation of the endoscope and the base of the positioning arm making it difficult to manipulate the draped end effector.

Accordingly, improved aseptic medical devices/components for visualization systems used in the surgical procedures to overcome the aforementioned problems by providing more intuitive, innocuous, and faster visualization adjustments and operations are needed.

### SUMMARY OF THE INVENTION

The foregoing needs are met, to a great extent, by the present invention, wherein in some aspects of embodiments of the invention are intended to address one or more of the above noted fundamental problems associated with draping systems for visualization systems used in conventional minimally invasive surgery. The improved draping methods and aseptic joint assembly of the various embodiments of the invention are applicable to many types of minimally invasive surgery, for example in the areas of thoracoscopic, laparoscopic, pelviscopic, arthroscopic surgeries. For laparoscopic surgery, significant utility will be found in cholecystectomy, hernia repair, bariatric procedures (bypass, banding, sleeve, or the like), bowel resection, hysterectomy, appendectomy, gastric/anti-reflux procedures, and nephrectomy.

According to some aspects of the disclosure, an aseptic joint assembly for a surgical visualization system having a positioning arm, an imaging device, and a display is disclosed. In particular, the aseptic joint assembly including an adaptor having a first locking mechanism used to removably attach the adaptor onto an end of the positioning arm of the surgical visualization positioning system, and a second locking mechanism used for attaching the adaptor to a hinge or rotating joint that is configured to rotate about an axis and either directly or indirectly support at least one or both of the display and the imaging device; and a first sterile drape bonded to at least a portion of the adaptor and configured to cover at least a portion of the positioning arm of the surgical visualization system.

In some embodiments, the aseptic joint assembly can include a second adaptor connected to the hinge or rotating joint and including one or both of a retaining receptacle for securing an imaging device's optical path onto the aseptic joint assembly and a retaining structure for securing a cannula onto the aseptic joint assembly. In addition, the aseptic joint assembly can include a second sterile drape, bonded to the second adaptor or a cap integrated with the second adaptor, used to cover at least a portion of the imaging device. The second sterile drape may include an opening for a percutaneous imaging device to be able to pass through and an adhesive/fastener to secure the sterile drape away from the surgical area.

A cover surrounding at least the portion of the adaptor on which the first sterile drape is bonded onto, wherein the first sterile drape is a sterile tubular drape that can be contained within the cover and pulled out to cover all around a length of the surgical visualization positioning system's arm may also be included in some embodiments. The sterile tubular drape can include an adhesive or fastener used to secure the distal end of the sterile tubular drape along a proximate section or the proximate end of the surgical visualization positioning system's arm. In some embodiments, the surgical visualization positioning system's arm is a generally curvilinear shaped prismatic arm and the sterile tubular drape can be fastened onto one or more sections of the generally curvilinear shaped prismatic arm by one or more of an adhesive of the sterile tubular drape and clips to avoid intended motion interference.

In accordance to additional aspects of the disclosure, the aseptic joint assembly can include a disposable adaptor having a first locking mechanism used to removably attach the adaptor onto a generally curvilinear shaped prismatic arm of the surgical visualization positioning system, a second locking mechanism used for attaching the adaptor to a second disposable adaptor that is configured to, directly or indirectly, support at least one or both of a display and an imaging device; and a cover surrounding at least a portion of the disposable adaptor and having a first end of a sterile tubular drape bonded thereto and wherein a second end of the sterile tubular drape is expandable to cover at least a portion of the curvilinear shaped prismatic arm.

Similarly, one or both of the first locking mechanism and the second locking mechanism is configured to be a quick release locking mechanism that can provide rotation between two attached parts along an axis. A second joint or hinge between the first disposable adaptor and the second adaptor providing rotational motion along an axis perpendicular to the rotation provided by the quick release locking mechanism may also be included. The second adaptor connected to the hinge or rotating joint includes one or both of a retaining receptacle for securing an imaging device's optical path onto the aseptic joint assembly and a retaining structure for securing a cannula onto the aseptic joint assembly. A second sterile drape, bonded to the second adaptor or a cap integrated with the second adaptor, used to cover at least a portion of the imaging device may also be included in some embodiments. The second sterile drape includes an opening for a percutaneous imaging device to be able to pass through and an adhesive/fastener to secure the sterile drape away from the surgical area. The sterile tubular drape includes an adhesive or fastener used to secure the distal end of the sterile tubular drape along a proximate section or the proximate end of the surgical visualization positioning system's arm.

Other aspects of the invention will be understood by those of skill in the art upon review of the teachings herein. Other aspects of the invention may involve combinations of the above noted aspects of the invention. These other aspects of the invention may provide various combinations of the aspects presented above as well as provide other configurations, structures, functional relationships, and processes that have not been specifically set forth above.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention.

FIG. 1A is a side view of an exemplary imaging device which may implement an aseptic joint assembly according to aspects of the disclosure.

FIG. 1B is a side view of an exemplary percutaneous cannula device which may implement an aseptic joint assembly according to aspects of the disclosure.

FIG. 1C is a cross section of a patient's skin is shown with the exemplary imaging device of FIG. 1A positioned inside the exemplary percutaneous cannula of FIG. 1B.

FIG. 2 is a perspective view of an exemplary embodiment of a positioning arm, a surgical table, an imaging device, and a patient lying on the surgical table.

FIG. 3A is a side view of the exemplary steering device being aimed down towards the patient's feet.

FIG. 3B is a side view of the exemplary steering device being aimed towards the center and towards the patient's back.

FIG. 3C is a side view of the exemplary steering device being aimed up towards the patient's head.

FIG. 4A is a bottom view of the steering device with the imaging device aimed towards the right of the patient.

FIG. 4B is a bottom view of the steering device with the imaging capturing device aired towards the left of the patient.

FIG. 5 is an exemplary surgical visualization system with an integrated aseptic joint assembly according to aspects of the present disclosure.

FIG. 6 is an exploded view of the exemplary integrated aseptic joint assembly of FIG. 5.

FIG. 7 is a cross section an exemplary assembled locking mechanism for the aseptic joint assembly.

FIG. 8 is the cross section of FIG. 7 showing the partially assembled locking mechanism.

The present invention is further described in the detailed description that follows.

### DETAILED DESCRIPTION OF THE INVENTION

Going forward, various aspects of the steering device of the present disclose may be illustrated by describing components that are coupled, attached, and/or joined together. As used herein, the terms “bonded”, “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present.

Relative terms such as “lower” or “bottom” and “upper” or “top” may be used herein to describe one element's relationship to another element illustrated in the drawings. It will be understood that relative terms are intended to encompass different orientations of the steering device in addition to the orientation depicted in the drawings. By way of example, if aspects of the steering device shown in the drawings are turned over, elements described as being on the “bottom” side of the other elements would then be oriented on the “top” side of the other elements. The term “bottom” can therefore encompass both an orientation of “bottom” and “top” depending on the particular orientation of the apparatus.

Various aspects of the steering device may be illustrated with reference to one or more exemplary embodiments. As used herein, the term “exemplary” means “serving as an example, instance, or illustration,” and should not necessarily be construed as preferred or advantageous over other embodiments of a steering arm or assembly disclosed herein.

Glossary

In this description and claims directed to the disclosure, various terms may be used for which the following definitions will apply:

“Articulated motion”, as used herein, can refer to the different parts of the steering device that allow rotation and/or translational motion up to a pre-configured degree of freedom via the end-effector. For example, the motion provided by each of the joint assemblies and the curvilinear prismatic joint in a frame about the surgical site.

“Aseptic joint assembly”, as used herein, refers to a sterile structure used to connect, either directly or indirectly, one or both of a display and an imaging device to an arm of a visualization system. For example, the aseptic joint may be/include a disposable connector with at least one, and preferably two locking mechanisms. In some embodiments, the aseptic joint can be configured with an expandable sterile drape to cover at least a portion of the arm in a manner that does not interfere with moving parts of the visualization system nor interfere with a physician performing a procedure or viewing a display in the surgical field. In addition, the aseptic joint may include one or more hinge joints and/or ball and socket joints that allow movement of the imaging device in relation to the arm of the visualization system. In some embodiments, the movement may be rotational movement on the hinge joint(s)′ axis and controlled by an end effector. The end effector may additionally operate a breaking system that can restrict the rotational movement of the aseptic joint assembly.

“Home position”, as used herein, can refer to a known and fixed location on the basic coordinate axis of the image capturing device manipulator where it comes to rest, or to an indicated zero position for each axis. In some embodiments, a unique position may be provided for each of various modes/settings that the steering device can be set to.

“Frame”, as used herein, can refer to a coordinate system in the sterile field used to determine a position and orientation of the image capturing manipulator in space, as well as the steering device's position within its model and in relation to the patient and/or the surgical area.

“Joint interpolated motion”, as used herein, can refer to the coordination of the movement of the joints, such that all joints arrive at the desired location simultaneously. In some embodiments, predictable paths that do not interfere with the line of sight of the surgeon and/or the surgical tools during surgery.

“Curvilinear prismatic joint”, as used herein, refers to a circular or generally arched joint that can provide linear sliding movement between two connected bodies. In particular, the curvilinear prismatic joint shape is designed to provide linear movement that by its design contours around a patient's body that is laying on a surgical bed. According to some aspects of the disclosure, the curvilinear prismatic joint can include a braking system that controls the linear movement of the two connected bodies.

“End effector”, as used herein, refers to a tool specifically designed to enable the steering device to perform the intended task of positioning an image capturing device during MIS.

“Sensor”, as used herein, can refer to one or more input devices used to enable a change in position or the fixing of a position of the manipulator relative to the surgical site by sending a resulting signal or data to at least one or more actuator and/or controller. The sensor(s) used may include sensors that respond to physical stimuli (such as heat, light, sound, pressure, magnetism, motion).

“Manipulator”, as used herein, can is a component of the steering arm which is configured to hold the image capturing device and, via the series of rotating and sliding joints, move the position of the image capturing device relative to the surgical site. The control of the manipulator may be by an operator via the end-effector and/or a programmable controller or any logic system (e.g., wired system).

“Imaging device” or “image capturing device”, as used herein, refers to a percutaneous optical visualization device or system, including for example, a percutaneous optical channel device, an endoscope, etc.

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as one skilled in the art would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure, which is defined solely by the appended claims and applicable law. Moreover, it is noted that like reference numerals represent similar parts throughout the several views of the drawings.

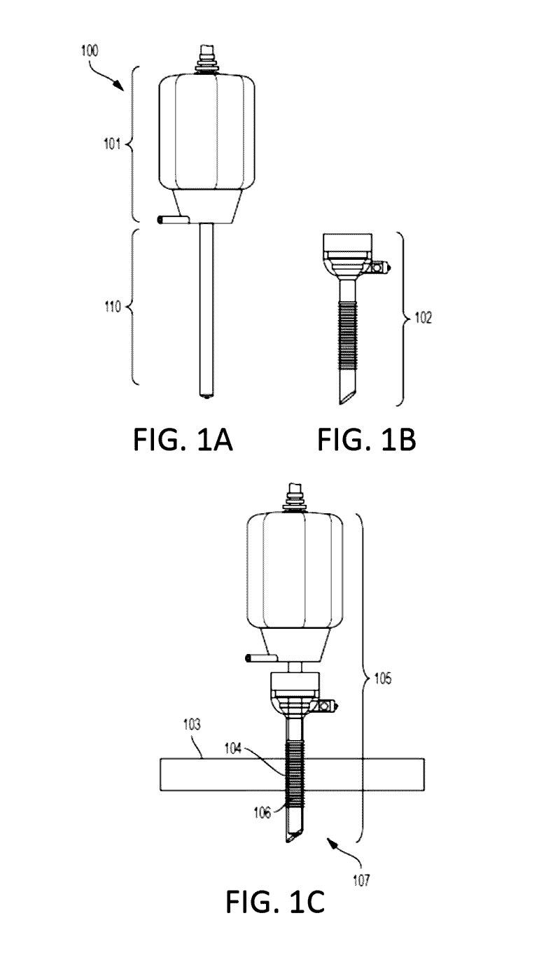

As previously mentioned, while surgical percutaneous visualization systems can vary greatly in complexity and implementations, they all must be kept sterile. In doing so, it is of upmost importance that parts of visualization systems remain functional, do not jeopardize safe and quick removal of parts in case of emergencies, and can be operated quickly and innocuously. Visualization systems may include conventional active or passive systems as well as systems described in co-pending U.S. application Ser. No. 14/727,023, filed, Jun. 15, 2015 and Ser. No. 14/011,493, filed Aug. 27, 2013, the complete disclosures of which are incorporated herein by reference. In addition, without limiting the scope of the disclosure, FIGS. 1-4 are included and described below, as an exemplary visualization system, and in order to provide context to the implementation and use of the different aspects of the present disclosure.

Referring now to FIG. 1A, a side view of an exemplary imaging device is shown. The imaging device 100 may be, for example, an endoscope which can include a tube/optical channel 110 ranging from 1 mm-20 mm that traverses the patient's skin through an incision to image a surgical site. In this exemplary embodiment, the optics inside can include a distal lens stack followed by a series of relay lenses to bring light from the surgical site to the image capturing device 101 attached proximally as shown. The imaging device 100 can include other optical components and at least one camera to digitally capture images and send them to one or more electronic displays, with at least one preferably being in the line of sight of the surgeon and in the sterile field, allowing the practitioner to perform MIS standing next to the patient as he/she would in open surgery.

The optical channel 110 can pass through a cannula 102 (shown in FIG. 1B), which holds the incision open and can allow free movement of the optical channel 110 portion to be introduced and removed from the surgical site. The cannula 102 may include a plurality of valves and seals to can allow surgical devices to be introduced and removed whilst avoiding the loss of insufflation gases from the internal surgical site. In some embodiments, the cannula 102 may be in logical communication with a user interface (not shown) of some steering device embodiments, to also lock the imaging device 100 prevent it from translating inside/outside of the body.

Referring to FIG. 1B, a side view of an exemplary percutaneous cannula device 102 which be implemented in a MIS visualization system. In FIG. 1C, the cannula 102 is shown with the optical channel 110 of the imaging device 100 inserted through the cannula 102 to gain access to the surgical site 107 below the skin 103. The imaging device 100, optical channel 101 and cannula 102 as shown form an imaging device 105. In some embodiments, the cannula can include a series of ribs 106 or other protruding external features that assist fixing the endoscopic imaging device in the incision 104.

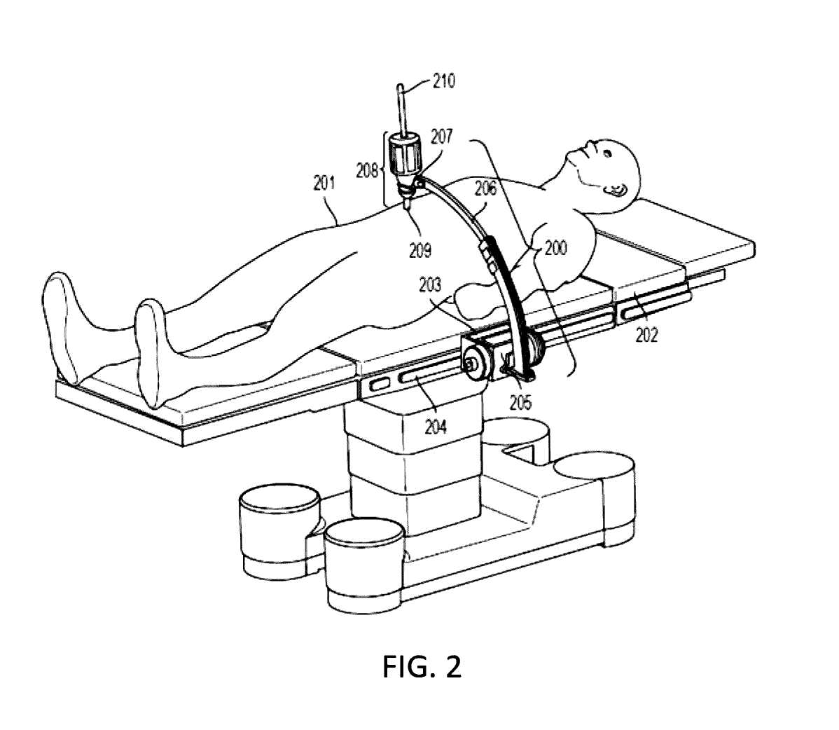

Referring now to FIG. 2, a perspective view of an exemplary embodiment of a positioning arm 200, a surgical table 202, the imaging device 100, and a patient 201 lying on the surgical table 202 is depicted. In particular, it depicts the imaging device 100 being positioned above a patient 201 lying on the surgical table 202 by the positioning arm 200. The positioning arm 200 includes a base 203 that is shown attached to a bedrail 204 of the surgical table 202. Attached to the base 203 can be a two degree of freedom gimbal 205. As shown starting from the base 203, the first rotational joint of the gimbal 205 can be parallel to bed rail 204. The second rotational axis can be orthogonal to the first and attached in serial. Attached to the gimbal can be curvilinear prismatic joint, e.g. a circular prismatic joint 206. These three joints in concert can allow positioning of the imaging device in Cartesian coordinates.

Further, the curvilinear prismatic joint can allow for large radius rotational motion about a point in a frame above the patient 201 and the surgical table 202. This point is adjustable depending on the pose of the gimbal. Using this configuration various safety and practical advantages are provided over conventional rotational joints that do not restrict the degrees on freedom and are not compatible with the surgical configuration. Moreover, the use of a linear prismatic joint would not be suitable as it would interfere with the patient and/or the surgical table.

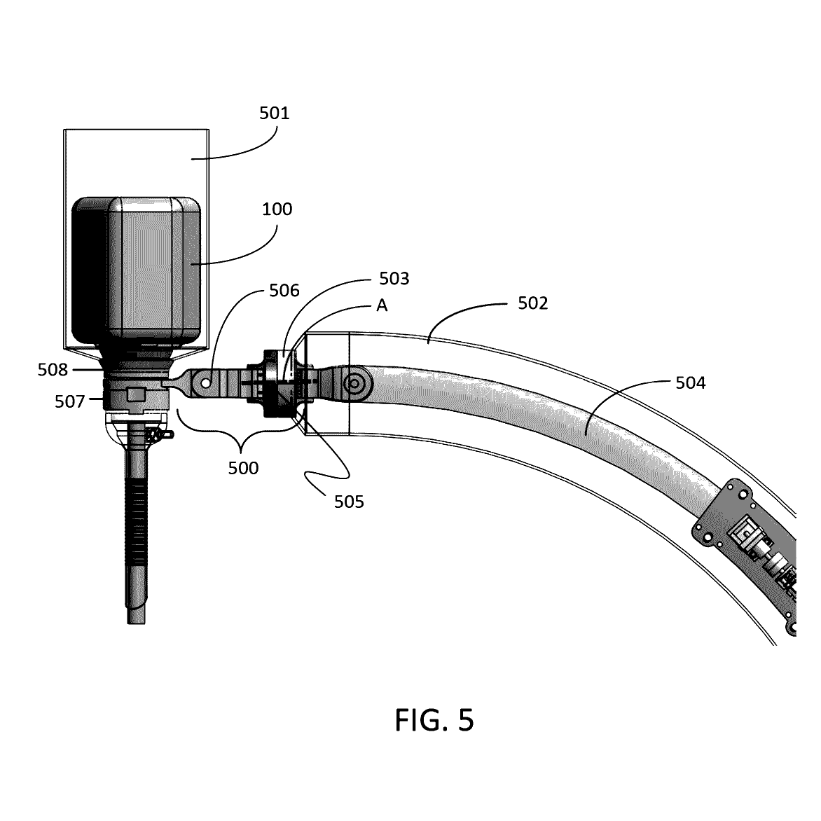

During MIS, it can be desired that the surgeon or assistant be able to rotate the imaging device 100 about a surgical incision 104 to achieve the correct pose of the imaging device 100. This may be necessary to ensure imaging of the, and/or accessibility, to specific locations of the surgical site and can be achieved in part by adding an additional two degrees of freedom via two rotational joints 207 at the end effector of the steering device. In accordance with some aspects of the disclosure further described in the description of FIGS. 5-8, one or two rotational joints 207 may be provided by the aseptic joint assembly 500.

Generally, the requirement of these two degrees of freedom is that its constituent rotational joints 207 are oriented such that they do not become parallel to the optical axis of the imaging device 100 during surgery. Ideally the two joints stay as dose to orthogonal as possible. The reason for this is the imaging device 100 is free to rotate in the cannula, resulting in a kinematic singularity if a parallel condition occurs. According to some aspects, the angle of rotation of the joints will be limited to less than 90 degrees from a nominal position where the imaging device can be oriented perpendicular to the surgical table (from a table top perspective). The angle of rotation can be limited, for example, by retaining structure that is fixed or by a part capable of being actuated by a controller (not shown) configured to control the range of motion of the system.

During use, the end effector 210 of the exemplary positioning arm 200 can be attached to some location away from the incision. This could be higher on the length of the imaging device 100, to an image recording device 101, or a coupler attached to the posterior end of the imaging device, for example. In some embodiments, the imaging device 100 and/or image recording device 101 may be ergonomically configured and include sensors to act as the end effector 210, thus eliminating the need for this additional part. The surgeon or assistant may use the end effector 210 to pivot the imaging device 100 about the incision until the desired pose is achieved. Once the desired pose is achieved, the gimbal 205 and circular prismatic joint 206 can lock via a braking system.

If the cannula 102 is sufficiently constrained by the incision 104, the imaging device 100 will be fully constrained and considered “held”. However, if the cannula 102 is not sufficiently constrained, the end effector joints 207 must also lock to fully constrain the imaging device 100 and consider it held. In practice the cannula 102 may be sufficiently constrained by friction between the incision 104 and the cannula 102 external features 106 and thus locking of 207 can be unnecessary.

The surgeon or assistant typically manipulates the imaging device by grasping the manipulator 210, which may be part of the imaging device 100/imaging recording device 101, with a hand. In some embodiments, the sensors to lock and unlock the arm may be part of the manipulator 210. The use of capacitive sensors would make the sensors invisible to the surgeon or assistant, making the use of the imaging device steering device almost—if not completely—innocuous to the surgeon or assistant. Capacitive sensors can include any sensor(s) used to detect and measure proximity, position or displacement, humidity, fluid level, and acceleration, for example, known in mouse track pads, touch displays, automotive door handles, industrial fluid indicators, etc. Other/additional sensors that may be desired in some embodiments can include sensor(s) that respond to physical stimuli (such as heat, light, sound, pressure, magnetism, motion) or a signal from a patient monitoring device. A signal from a patient monitoring device may include, for example, a signal relating to the patient's heart rate received by the controller.

In yet additional embodiments, additional safety sensors may be positioned in joints 207, 203, 205, for example, to limit or provide a warning when the range of motion approaches an unsafe position. In some embodiments, the two capacitive sensors may be located on opposite sides of the manipulator 210 to eliminate inadvertent unlocking of the arm due to bumping. Logic circuitry ensure the arm only unlocks when multiple sensors on the manipulator 210 are activated ensures that the arm is unlocking due to a grab event versus a bump event. In additional embodiments, a small vibration device (motor) and/or light may be included in the manipulator 210 to provide a warning to the user about a condition. A condition may include, for example, a heart rate electrical signal falling outside a predetermined threshold, an electrical signal received from the imaging device's sensor located on the distal end sensing the distance to a delicate boundary (e.g. organ), etc.

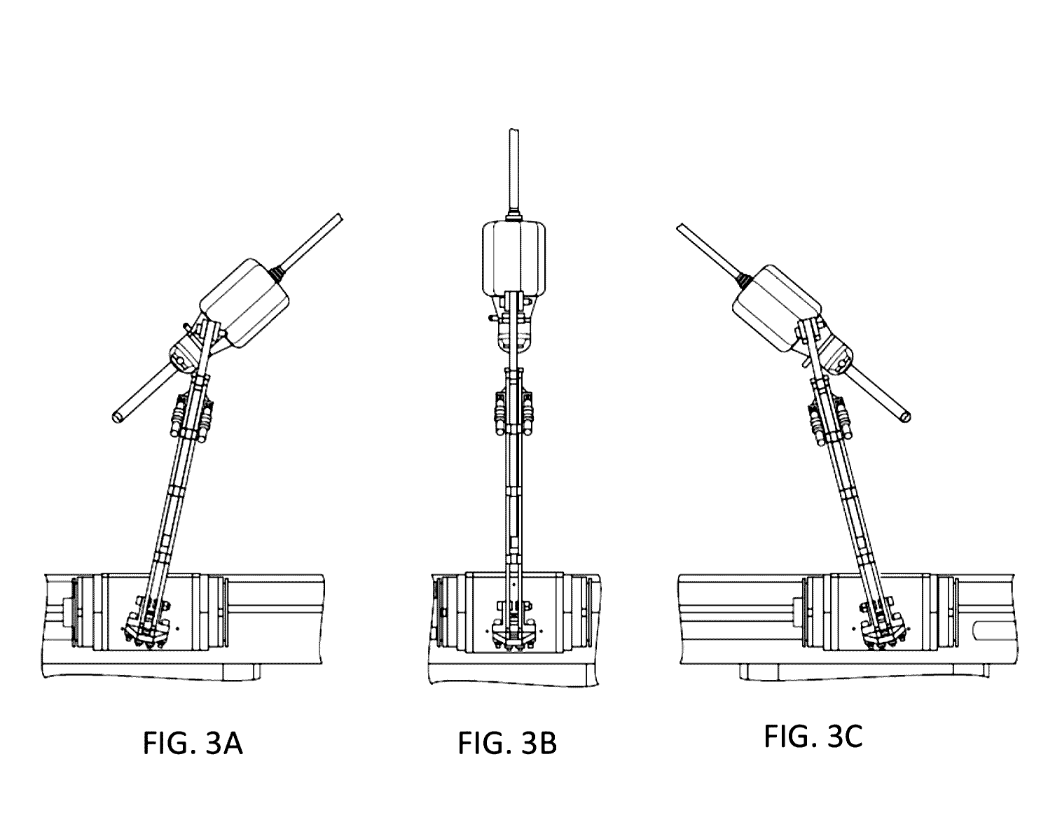

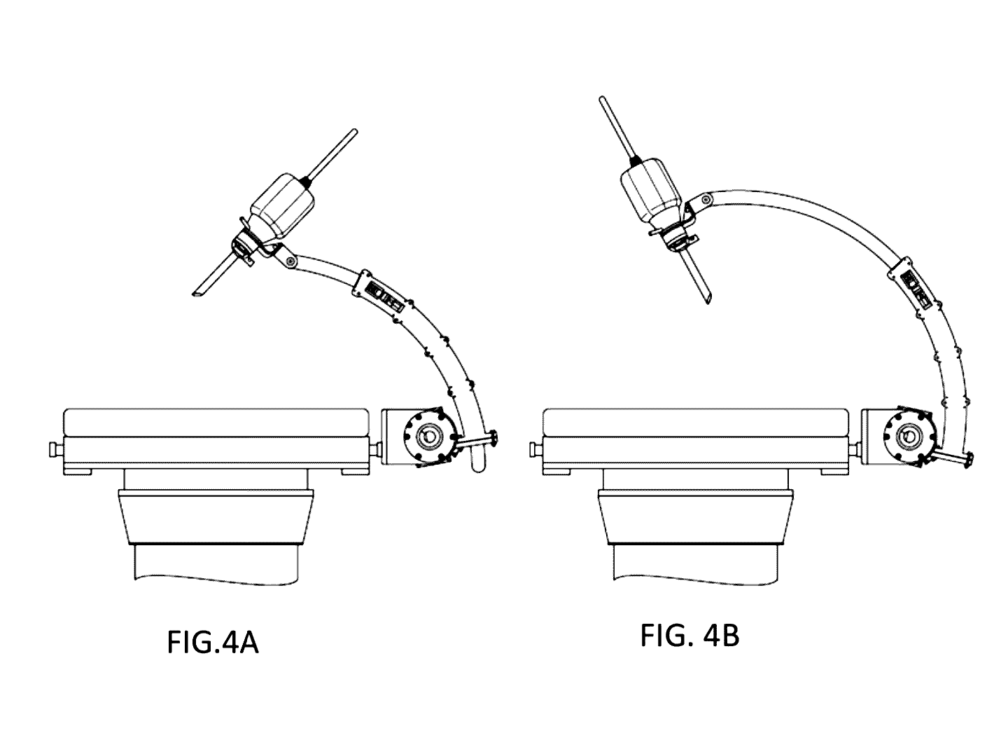

FIGS. 3A-3C and 4A-4B are shown to demonstrate the importance of providing solutions for aseptic visualization systems that do not interfere with the controlled movement of the systems. More specifically, these figures are used to demonstrate why the methods and/or devices used to keep the surgical visualization systems sterile must not affect practicality and remain innocuous to the system so that an imaging device can be positioned at different locations above a surgical site. Referring to FIG. 3A, FIG. 3B, and FIG. 3C, side views of the exemplary positioning arm are shown where the imaging system is rotated around a fixed incision point in which the cannula may be inserted. FIG. 3A shows the endoscopic imaging system pointing “down” towards the location of the patient's feet. FIG. 3B shows the endoscopic centered pointing at the patient's back. FIG. 3C shows the endoscopic imaging system “up” towards the patient's head. As shown in FIGS. 3A-3C, in some embodiments, different parts may be configured to move in concert to produce the desired joint interpolated motion to tilt about the incision and provide the user the ability to look up and down in the surgical field. Because of the intended ability of the systems to move up and down about a fixed point, the use of conventional surgical drapes to keep system sterile is inadequate.

Referring now to FIG. 4A is a side bottom view of the steering device with the imaging device aimed towards the right of the patient. In particular, showing the endoscopic imaging system pointing “right” towards the location of the patient's right side. Referring now to FIG. 4B, the endoscopic imaging system pointing “left” towards the location of the patient's left side. According to some embodiments, these figures show how the circular prismatic joint and the gimbals can provide joint interpolated motion enabling them to move in concert and produce the desired tilting motion about the incision to look left and right in the surgical field. Similarly because of the intended ability of the systems to move left and right about a fixed point, the use of conventional surgical drapes to keep system sterile is inadequate.

Referring now to FIG. 5, an exemplary surgical visualization system with an integrated sterile aseptic joint assembly 500 according to aspects of the present disclosure is depicted. In particular, an integrated aseptic joint assembly 500 is shown connecting a positioning arm's distal end 504 and an imaging device 100. According to some aspects, the aseptic joint assembly 500 can include an adaptor 505 that serves at least in part to connect a coupling structure to the positioning arm's 500 distal end 504. In some preferred embodiments, the distal end 504 of the positioning arm 200 can also include a complementary structure to firmly connect the adaptor 505 to the positioning arm 200. The connection should be a tough connection that can allow for it to be disconnected after use or in the case of an emergency. In some embodiments, a retaining feature, e.g. a pin (not shown), of the coupling structure of the adaptor 505 may be configured to purposely break after removal to prevent reuse and ensure an aseptic environment is kept.

In some embodiments, a cap 503 can surround/be integrated with the adaptor 505. The implementation of a cap can be particularly useful, for example, in embodiments in which the adaptor 505 is designed to be sterilized and reused. More importantly, the cap 503 may include a surgical drape 502 bonded thereto. The surgical drape 502 can be, for example, a tubular sterile barrier that can cover contaminated surfaces of the surgical visualization system's positioning arm 200. According to some aspects, the surgical drape 502 should be resistant to punctures and tears to prevent microbial contamination of the sterile field. Moreover, this sterile barriers is preferably plastic and disposable after use, and/or may contain any other material such as a treated cloth or sheet of fabric that can provide a sterile barrier and is resistant to fluid penetration, lint free, flame resistant, etc.

According to some embodiments, the surgical drape 502 is expandable and before use can be collapsed into the cap 503 so that it can be easily replaced. According to yet additional embodiments, at least a portion of the interior surface of the surgical drape 502 can include an adhesive that can be used to secure the surgical drape 502 to a portion of the positioning arm 200 so that it does not fall/slide when the system is being used. The adhesive may be found at different pre-determined portions along the length of the surgical drape 502 and at the end. In those embodiments, the adhesive may be included so that the surgical drape 502 is only glued in portions that do not interfere with moving parts of the positioning arm 200. For example, this can be accomplished by leaving additional length of the surgical drape 502 in expandable positioning arm's sections 504. Alternatively, the adhesive can be replaced with one or more clips (not shown) that can placed on the positioning arm 200. In some embodiments, the clips may slide along a length of the positioning arm 200 when it is extended, or rotating, similarly preventing the surgical drape 502 from getting tangled and interfering with the controlled movements.

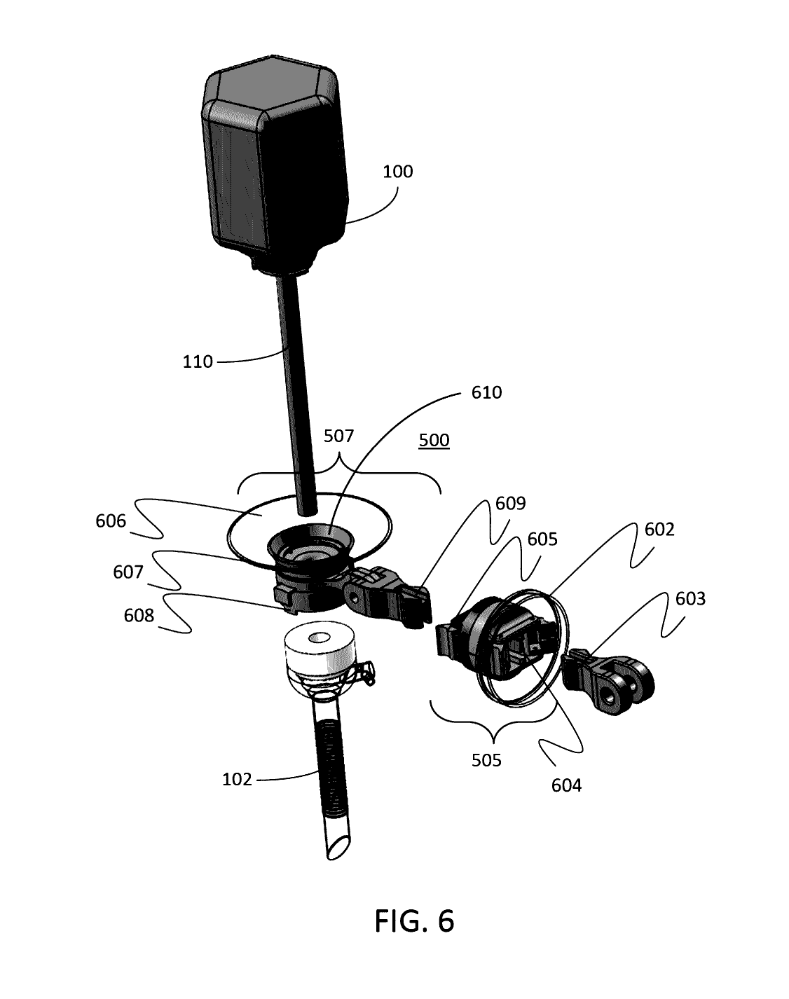

The adaptor 505 may include or be attached to a first rotating feature/joint that can allow rotating motion along an axis (shown by dotted line A) perpendicular to the positioning arm 200. In one embodiment, by means of the locking system, the rotating joint may be formed between the adaptor 505 and the positioning arm's distal end 504 (as shown in FIGS. 6-8. In alternative embodiments, a separate intermediate rotating joint is included. The rotating feature may be disposable or sterilized after use. Accordingly, the rotating feature/joint can allow the visualization system towards the lower end of the surgical site and up towards the upper end of the surgical site by manipulation of an end effector, as shown in FIGS. 3A-3C.

Moreover, in some embodiments, the adaptor can be connected to a second rotating joint 506 that can allow rotating motion along a Y-axis. Likewise, rotating joint 506 may be disposable or sterilized after use. According to some aspects, the rotation provided by one or both feature/joint and joint 506 can be controlled and/or limited. For example, the degrees of rotation can be limited to 120 degrees and more preferably to 90 degrees. In some embodiments, rotating feature/joint and joint 506 may be replaced by a single ball joint or a joint that provides rotation about two points/axis. Moreover, in some embodiment variations that may be within the scope of the disclosure, only one or no rotating joints may be included in the aseptic joint assembly 500.

In some embodiments, the second rotating joint 506 may be connected to or include a second adaptor 507. Adaptor 507 may be fixed or removably fixed to a coupling structure of a cannula 102 and/or the imaging device 100. Optionally a second cap 508 may be affixed to the adaptor 507 which can also include a similar surgical drape 501 used to cover one or both the imaging device 101 and the display (not shown). According to some aspects, FIG. 5 shows the nominal position of 505 with the optical axis of 100, the rotational axis and the rotational axis of 506 all orthogonal and in which the kinematics of the positioning device can behave the best. This relative positioning of the joints 505 and 506 can also be switched depending on the requirements of the surgery. For example, the imaging device 100 can rotate as shown in FIGS. 3A-3C about joint 505 without the orthogonality of joint 506 changing. Thus if the pose changes dramatically in this direction—large angles—the joints of the aseptic joint assembly 500 can stay well behaved. However if 505 is rotated as shown in FIG. 3 about joint 505 to +−90 degrees, the optical axis and joint 506 may become parallel. Thus relative position of joints 505 and 506 can be chosen based on the positioning arm mount location and the surgical requirements to avoid such kinematic singularities.

In some embodiments, additional couplers or adaptors may be attached to/contained by the aseptic joint assembly. For example, some embodiments may include a third adaptor used to support a display (not shown). The third adaptor may also include a surgical drape which may further be configured with a cap, as previously described. Size and configuration of the surgical drape 501 and any additional drapes incorporated to be functional with the aseptic joint assembly may vary and not be cylindrical. However, these drapes should be sterile include clips and adhesive portions to minimize the probability of entanglement resulting in interference with the controlled movement and/or posing a hazard in some situations.

Aseptic joint assembly materials may include for example, an acetal copolymer, high-density polyethylene, polypropylene, polycarbonate, acrylic, stainless steel, surgical grade steel, and the such. In some embodiments, for example, only the caps and drapes may be disposable after use and the rotating joints and adaptors may removable for sterilization and reuse. Accordingly, reusable parts may be made of the lower costs plastics and reusable serializable parts may be made of stainless steel, surgical steel, graphite, and the such. In some embodiments, surgical drapes and/or parts of the aseptic joint assembly, imaging device, and end effector may include antimicrobial nano-pattern surfaces/coatings. For example, these may be found near the surgical site in hard to cover structures and sensors that need to be exposed.

Referring now to FIG. 6, an exploded view of the integrated aseptic joint assembly 500 is shown. In particular, the individual parts that may be implemented according to aspects of the disclosure are shown with an imaging device 100 as it may be intended for assembly. According to some aspects, adaptor 505 is shown including structural coupling structure 604 being complementary to an exemplary structure 603 which may be located or fixed on the distal end 504 of the positioning arm 200. The cap 503, unlike FIG. 5, shows the collapsed and contained within surgical drape 602. This surgical drape 602 can expand as shown in FIG. 5 at 502 and extend along a sterile length of the positioning arm 200.

In some embodiments, a locking structure, for example, as provided by structure 609 and 605 can provide connection from the adaptor 505 to adaptor 507, which may include cap 508 and the appropriate surgical drape 606 shown collapsed within the cap 508 in FIG. 6. Moreover, as shown in FIG. 6, a retaining structure can be included in adaptor 507, e.g., clips 608, to the proximate end of the cannula 102 to the aseptic joint assembly 500. According to some aspects, an optical path 110 of the imaging device 100 may also be disposed within adaptor 507.

The surgical drapes are shown folded/collapsed at 606 and 602 to allow attachment of the disposable arm adapter 505 to the positioning system imaging device 100, which in some embodiments may serve as an effector of the positioning device. One skilled in the art will recognize from the teachings of this disclosure that a connection between the aseptic joint assembly and the end effector is needed whether directly or indirectly but is not limited to a connection to the imaging device 100.

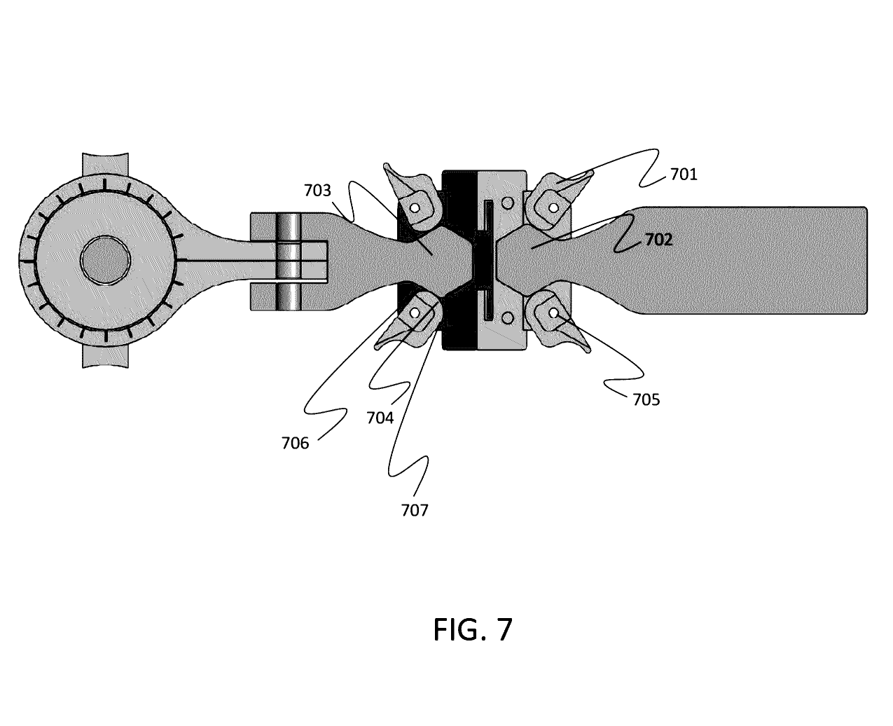

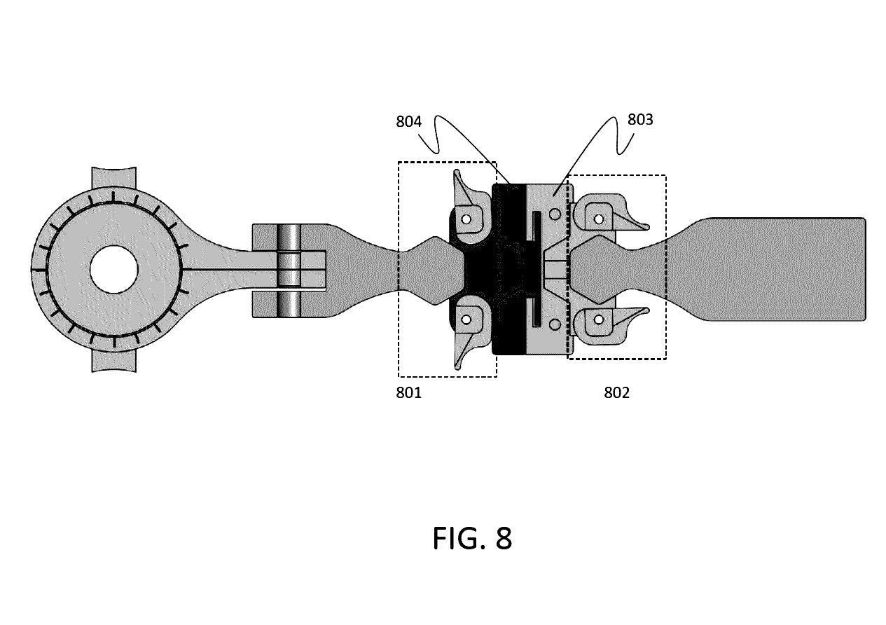

Attaching means between joints and adaptors can be by a novel quick release cleat mechanism further explained in FIGS. 7 and 8. As previously mentioned, attaching means between the end effector (in this case the imaging device 100) and the adapter 507 may be by insertion of an optical path having a hexagonal shaped cross section that fits into retaining receptacle 604. According to some aspects, this receptacle can include spring loaded retaining features that allow the end effector to enter the receptacle by swinging out of the way but by design do not allow the optical path to pull out of the retaining receptacle 604 during normal use unless an actuator (not shown) is pressed, for example. In other embodiments, the optical path can be released by manually rotating the retaining features out of the way. As such, the aseptic joint assembly can quickly be attached onto the surgical imaging system and covered with the surgical drape 502 and, in some embodiments, any additional surgical drapes, such as surgical drape 501.

Referring now to FIG. 7, a cross section of an exemplary assembled locking mechanism for the aseptic joint assembly is shown. In particular, a quick release cleat mechanism is shown in the locked position. Cross sectioned retaining features 701A and 701B are shown engaged with hexagonal cross sections 702 and 703 of the quick release components 603 and 609.

The key to the function of the quick release component is the surface profile 704. The distance from the rotating pin 705 to the surface 704 increases from the top of the retaining feature 706 to the bottom of the retaining feature 707. This means that as 603 and 609 are pulled out of the quick release mechanism, friction between the components will rotate the retaining feature causing the surface 704 to mechanically interfere with 603 and 609 making removing 603 or 609 impossible by pulling on them alone modulo structural failure. Accordingly, to release 603 and 609 the retaining features 701 must be externally rotated until the hexagonal profiles 702 and 703 clear the retaining features.

Referring now to FIG. 8, the cross section of FIG. 7 showing the partially assembled locking mechanism is shown. Configuration 801 shows the hexagonal cross section 702 before engaging with the retaining cleat 604. Here the retaining features 707 are rotated to the most engaging orientation by return springs (not shown). As the hexagonal cross section 702 is pushed into the retaining cleat 604, the retaining features 707 swing out of the way as shown in 802. To remove 702, the retaining features 707 must be manually moved to the configuration in 802. This provides the quick attachment and quick release features of the system.

FIG. 8 also shows the interlocking rotational joint of the sterile disposable arm adapter 601. The left component 804 is interlocked with the right component 803 such that the sterile disposable arm adapter 601 is rigid except for the rotational axis.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

We claim:

1. An aseptic joint assembly for a surgical visualization system having a positioning arm, an imaging device, and a display, the aseptic joint assembly comprising:
an adaptor having;
a first locking mechanism used to removably attach the adaptor onto an end of the positioning arm of the surgical visualization positioning system, and
a second locking mechanism used for attaching the adaptor to a hinge or rotating joint that is configured to rotate about an axis and either directly or indirectly support at least one or both of the display and the imaging device; and
a first sterile drape bonded to at least a portion of the adaptor and configured to cover at least a portion of the positioning arm of the surgical visualization system.

2. The aseptic joint assembly of claim 1, additionally comprising:
a second adaptor connected to the hinge or rotating joint and including one or both of a retaining receptacle for securing an imaging device's optical path onto the aseptic joint assembly and a retaining structure for securing a cannula onto the aseptic joint assembly.

3. The aseptic joint assembly of claim 1, additionally comprising:
a second sterile drape, bonded to the second adaptor or a cap integrated with the second adaptor, used to cover at least a portion of the imaging device.

4. The aseptic joint assembly of claim 3, wherein the second sterile drape includes an opening for a percutaneous imaging device to be able to pass through and an adhesive/fastener to secure the sterile drape away from the surgical area.

5. The aseptic joint assembly of claim 1, additionally comprising:
a cover surrounding at least the portion of the adaptor on which the first sterile drape is bonded onto, wherein the first sterile drape is a sterile tubular drape that can be contained within the cover and pulled out to cover all around a length of the surgical visualization positioning system's arm.

6. The aseptic joint assembly of claim 5, wherein the sterile tubular drape includes an adhesive or fastener used to secure the distal end of the sterile tubular drape along a proximate section or the proximate end of the surgical visualization positioning system's arm.

7. The aseptic joint assembly of claim 5, wherein the surgical visualization positioning system's arm is a generally curvilinear shaped prismatic arm and the sterile tubular drape can be fastened onto one or more sections of the generally curvilinear shaped prismatic arm by one or more of an adhesive of the sterile tubular drape and clips to avoid intended motion interference.

8. The aseptic joint assembly of claim 1, wherein the locking mechanism is a quick lock mechanism that allows for rotation of two joined parts along an axis perpendicular to the positioning arm.

9. An aseptic joint assembly for use in a surgical visualization positioning system, the aseptic joint assembly comprising:
an adaptor having;
a first locking mechanism used to removably attach the adaptor onto a generally curvilinear shaped prismatic arm of the surgical visualization positioning system,
a second locking mechanism used for attaching the adaptor to a second disposable adaptor that is configured to, directly or indirectly, support at least one or both of a display and an imaging device; and
a cover surrounding at least a portion of the adaptor and having a first end of a sterile tubular drape bonded thereto and wherein a second end of the sterile tubular drape is expandable to cover at least a portion of the curvilinear shaped prismatic arm.

10. The aseptic joint assembly of claim 9, wherein one or both of the first locking mechanism and the second locking mechanism is configured to be a quick release locking mechanism that can provide rotation between two attached parts along an axis.

11. The aseptic joint assembly of claim 10, additionally comprising:
a second joint or hinge between the first disposable adaptor and the second adaptor providing rotational motion along an axis perpendicular to the rotation provided by the quick release locking mechanism.

12. The aseptic joint assembly of claim 11, wherein the second adaptor connected to the hinge or rotating joint includes one or both of a retaining receptacle for securing an imaging device's optical path onto the aseptic joint assembly and a retaining structure for securing a cannula onto the aseptic joint assembly.

13. The aseptic joint assembly of claim 9, additionally comprising:
a second sterile drape, bonded to the second adaptor or a cap integrated with the second adaptor, used to cover at least a portion of the imaging device.

14. The aseptic joint assembly of claim 13, wherein the second sterile drape includes an opening for a percutaneous imaging device to be able to pass through and an adhesive/fastener to secure the sterile drape away from the surgical area.

15. The aseptic joint assembly of claim 9, wherein the sterile tubular drape includes an adhesive or fastener used to secure the distal end of the sterile tubular drape along a proximate section or the proximate end of the surgical visualization positioning system's arm.

16. The aseptic joint assembly of claim 9, wherein the sterile tubular drape can be fastened onto one or more sections of the generally curvilinear shaped prismatic arm by one or more of an adhesive of the sterile tubular drape and clips to avoid intended motion interference.

17. An aseptic joint assembly for use in a surgical visualization positioning system, the aseptic joint assembly comprising:
a first adaptor having;
a first locking mechanism used to attach the adaptor onto the end of an arm of the surgical visualization positioning system,
a second locking mechanism used for attaching the first adaptor to a second adaptor that is configured to, directly or indirectly, support at least one or both of a display and an imaging device; and
a surgical tubular drape bonded to either the first adaptor or a supplementary cap for the adaptor, wherein the surgical tubular drape is expandable to cover at least a portion of the arm.

18. The aseptic joint assembly of claim 17, wherein one or both of the first locking mechanism and the second locking mechanism is configured to be a quick release locking mechanism that can provide rotation between two attached parts along an axis.

19. The aseptic joint assembly of claim 18, additionally comprising:
a second joint or hinge between the first adaptor and the second adaptor providing rotational motion along an axis perpendicular to the rotation provided by the quick release locking mechanism.

20. The aseptic joint assembly of claim 19, wherein the second adaptor connected to the hinge or rotating joint includes one or both of a retaining receptacle for securing an imaging device's optical path onto the aseptic joint assembly and a retaining structure for securing a cannula onto the aseptic joint assembly and wherein at least a portion of a surface of one or more of the retaining receptacle, the retaining structure, and the second adaptor or rotating joint includes an antimicrobial nano-patterned surface.
